(19) United States
(12) Patent Application Publication
(10) Pub. No.: US 2025/0388451 A1
(43) Pub. Date: Dec. 25, 2025

(54) SYSTEM FOR DISPENSING AND MANAGEMENT OF OIL AND METHOD EMPLOYED THEREOF

(71) Applicant: MANYA MEREDDY, CUMMING, GA (US)

(72) Inventor: MANYA MEREDDY, CUMMING, GA (US)

(21) Appl. No.: 19/311,134

(22) Filed: Aug. 27, 2025

(51) Int. Cl. B67D 3/00; G09B 5/02; G09B 5/04; G10L 15/22; G16H 20/60

(52) U.S. Cl. CPC B67D 3/0074; B67D 3/0003; B67D 3/0093; G09B 5/02; G09B 5/04; G16H 20/60; G10L 15/22; G10L 2015/223

(57) ABSTRACT

A system and method for dispensing and managing cooking oil are disclosed. The system comprises a dispenser unit with an oil reservoir, a sensing unit for measuring dispensed oil, a dispensing mechanism, a microcontroller, and a communication module for wireless data transmission. A user device, running a mobile application, receives usage data, processes it against predefined thresholds, and provides real-time feedback. A cloud server stores historical data, applies machine learning algorithms for personalized suggestions and alerts, and integrates with external health applications and smart kitchen appliances. This invention enables precise monitoring, intelligent management, and promotes healthier oil consumption habits through data-driven insights and seamless connectivity.

### COPYRIGHT AND TRADEMARK NOTICE

This application includes material which is subject or may be subject to copyright and/or trademark protection. The copyright and trademark owner(s) have no objection to the facsimile reproduction by any of the patent disclosure, as it appears in the Patent and Trademark Office files or records, but otherwise reserves all copyright and trademark rights whatsoever.

### TECHNICAL FIELD

The present invention relates generally to the field of liquid dispensing systems, and more particularly to a smart oil dispenser with an integrated system for monitoring and managing oil consumption.

### BACKGROUND

The present invention relates generally to the field of liquid dispensing systems, particularly those used in culinary and domestic environments. Within this domain, various devices exist for the storage and dispensing of cooking oils, ranging from simple pour bottles to more elaborate manual sprayers. These existing solutions primarily focus on the ease of pouring or application, aiming to provide a convenient means for users to add oil during cooking.

However, current cooking oil dispensers suffer from significant limitations that present challenges for modern kitchens and health-conscious consumers. Most conventional dispensers are entirely manual, lacking any form of smart features or connectivity. For instance, products like the FASTIVEN Oil Sprayer and PNB Kitchen mate's designs, while offering aesthetic or ergonomic benefits, are manual and do not incorporate any form of usage tracking. Similarly, existing literature does not address the functionality of smart dispensing or usage monitoring.

A critical gap in the existing market and prior art is the absence of a cooking oil dispenser that integrates with an application to track usage, provide real-time feedback, and offer warnings regarding excessive consumption. Current dispensers do not connect to apps, nor do they provide mechanisms for monitoring the quantity of oil dispensed. This lack of intelligent features means users often rely on estimation, leading to inconsistent oil usage, potential waste, and difficulty in adhering to dietary or health goals. There is a clear unfulfilled need for a system that can accurately measure oil dispensed, provide real-time alerts for overuse, and offer personalized insights based on user habits. Furthermore, the integration of features such as recipe-based oil suggestions, gamification to encourage healthier habits, connectivity with other smart kitchen appliances (like scales or ovens), and integration with health tracking applications represents a significant advancement over the current state of the art. The ability to incorporate learning algorithms to adapt oil suggestions based on individual user habits, or to offer voice control and accessibility features, further highlights the deficiencies of existing solutions.

In light of the existing problems and limitations, there is a compelling need for a novel smart oil dispenser that addresses these shortcomings by providing precise usage tracking, intelligent feedback, and seamless integration with digital platforms to promote healthier cooking habits and reduce waste.

### SUMMARY

The following invention presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

An objective of the present disclosure is to provide a smart oil dispenser system that accurately tracks oil usage and provides warnings to users regarding excessive consumption.

Another objective of the present disclosure is to enable seamless connectivity between the oil dispenser and a mobile application for real-time data monitoring and immediate user feedback.

Another objective of the present disclosure is to implement precise oil measurement mechanisms, such as weight sensors or flow meters, to ensure accurate usage tracking.

Another objective of the present disclosure is to incorporate learning algorithms that adapt oil dispensing suggestions based on individual user habits and preferences.

Another objective of the present disclosure is to facilitate integration with other smart kitchen appliances, such as smart scales and ovens, for automated and recipe-synchronized oil dispensing.

Another objective of the present disclosure is to enable integration with health tracking applications to monitor daily oil intake and suggest healthier oil alternatives based on user health goals.

Another objective of the present disclosure is to provide intuitive control mechanisms, including voice control, for hands-free operation of the dispenser.

Another objective of the present disclosure is to enhance user accessibility through features such as voice-guided instructions.

Another objective of the present disclosure is to implement gamification features to encourage reduced oil consumption and promote healthier cooking habits.

Another objective of the present disclosure is to include sustainability features, such as mechanisms for collecting and reusing excess oil, to minimize waste.

In an exemplary embodiment of the present disclosure, a smart oil dispenser is provided comprising a reservoir for holding cooking oil, a dispensing mechanism, and an integrated sensing unit. The sensing unit, which may include one or more weight sensors (e.g., load cells) positioned beneath the oil reservoir or flow meters integrated within the dispensing nozzle, is configured to precisely measure the quantity of oil dispensed. A processing unit within the dispenser receives data from the sensing unit and transmits this data wirelessly, via communication modules such as Bluetooth or Wi-Fi, to a connected mobile application. The mobile application is configured to receive and process the oil usage data, compare it against predefined or user-set thresholds, and generate real-time visual and/or audible warnings if excessive oil consumption is detected, thereby achieving the objective of tracking oil usage and providing warnings.

Another exemplary embodiment of the present disclosure focuses on seamless connectivity and real-time feedback. The smart oil dispenser includes a dedicated communication module (e.g., a Wi-Fi or Bluetooth Low Energy module) that establishes a secure and continuous connection with a user's smartphone or tablet running the proprietary mobile application. This connection facilitates the immediate transmission of dispensed oil quantities to the application, allowing for real-time display of usage statistics, graphical representations of consumption patterns, and instantaneous feedback or alerts to the user, directly addressing the objective of seamless connectivity and real-time data monitoring.

In another exemplary embodiment of the present disclosure, precise oil measurement is achieved through a combination of a high-resolution load cell placed beneath the oil container and a calibrated flow meter integrated into the dispensing spout. The load cell continuously monitors the weight of the oil in the reservoir, detecting minute changes as oil is dispensed. Concurrently, the flow meter provides a volumetric measurement of the oil flowing through the spout. Data from both sensors is cross-referenced by the dispenser's internal microcontroller to ensure highly accurate measurement of dispensed oil, which is then transmitted to the mobile application, thus fulfilling the objective of implementing precise oil measurement mechanisms.

Another exemplary embodiment of the present disclosure incorporates advanced learning algorithms within the mobile application. This algorithm analyzes historical oil usage data, including quantities, frequency, time of day, and user-inputted meal types or cooking methods. Based on these learned patterns, the application can dynamically adjust personalized oil suggestions for various dishes or cooking scenarios. For instance, if the user consistently uses less oil for a particular recipe, the algorithm will adapt its recommendations and warning thresholds accordingly, thereby achieving the objective of incorporating learning algorithms for adaptive oil suggestions.

Another exemplary embodiment of the present disclosure enables comprehensive kitchen integration. The smart oil dispenser system, through its mobile application, is configured to communicate with other smart kitchen appliances via a standardized protocol or APIs. For example, it can receive data from smart scales to automatically suggest or dispense precise oil amounts based on the weight of other ingredients. It can also interface with smart ovens to recommend or pre-set oil quantities for specific recipes or cooking programs being executed, thereby facilitating the objective of integration with other smart kitchen appliances.

In another exemplary embodiment of the present disclosure, the mobile application features an interface for integrating with third-party health and wellness applications. This integration allows the oil consumption data tracked by the dispenser to be automatically synchronized with the user's overall dietary intake records within their preferred health app. Based on the user's health goals and dietary preferences, the application can also provide suggestions for healthier oil alternatives (e.g., olive oil over less healthy options), directly addressing the objective of integrating with health apps to track daily oil intake and suggest healthier oils.

Another exemplary embodiment of the present disclosure focuses on intuitive user interaction through voice control. The dispenser or its associated mobile application is equipped with a microphone and voice recognition capabilities, allowing users to operate the dispenser hands-free. Users can issue commands such as “dispense 15 milliliters of oil” or “stop dispensing,” which are processed and executed by the system, thereby achieving the objective of providing intuitive voice control.

Another exemplary embodiment of the present disclosure enhances accessibility by incorporating voice-guided instructions. For users with visual impairments or those who prefer auditory cues, the mobile application and/or the dispenser itself can provide spoken instructions for setup, operation, and alerts. This includes verbal confirmations of dispensed quantities and spoken warnings about overuse, ensuring comfortable usage for a wider range of users, thus addressing the objective of adding accessibility features.

In another exemplary embodiment of the present disclosure, gamification elements are integrated into the mobile application. Users can participate in challenges, earn points or badges for consistently staying within recommended oil limits, or compete with friends or family members. The application provides progress tracking and visual rewards, transforming the act of managing oil consumption into an engaging and motivating experience, thereby achieving the objective of gamification to encourage healthier habits.

Another exemplary embodiment of the present disclosure includes sustainability features. The dispenser is designed with a dedicated, easily accessible collection tray or a return mechanism for collecting any excess or spilled oil. The system can detect when a certain volume of oil has accumulated in this tray and provide an alert via the app, suggesting options for responsible reuse (e.g., for certain cooking methods, if safe) or proper disposal, thereby fulfilling the objective of incorporating sustainability features.

### BRIEF DESCRIPTION OF THE DRAWINGS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

FIG. 1 depicts a mechanical structural block diagram of the smart oil dispenser. It illustrates the primary physical components of the dispenser, including the oil reservoir, the integrated sensing unit for measurement, the dispensing mechanism, the protective housing, and the power source. An optional user interface is also shown for direct interaction.

FIG. 2 depicts a comprehensive system integration block diagram of the smart oil dispenser. It illustrates how the dispenser's hardware components (sensing unit, MCU, communication module, actuator) wirelessly connect to a user's mobile device running the application. This application, in turn, interacts with a cloud server for advanced functionalities and further integrates with third-party health applications and other smart kitchen appliances.

FIG. 3 depicts an overall functional flow diagram of the smart oil dispenser system. It illustrates the sequence of operations from user interaction and oil dispensing to data transmission, processing by the mobile application, and subsequent cloud-based functionalities including data storage, learning algorithms, and integration with external services.

FIG. 4 depicts a sample mobile application main dashboard screen. It provides an overview of daily oil usage, real-time dispenser status, quick dispense options, and personalized suggestions, serving as the central hub for user interaction.

FIG. 5 depicts a sample mobile application dispensing control screen. This screen allows users to select dispensing modes (manual, recipe, voice), input desired oil quantities, and initiate or stop the dispensing process, providing real-time feedback during operation.

FIG. 6 depicts a sample mobile application usage history and analytics screen. It presents historical oil consumption data through charts, offers personalized health insights based on usage patterns, and displays progress in gamification challenges to encourage healthier habits.

FIG. 7 depicts a sample mobile application settings and integration screen. This screen allows users to customize app preferences, manage integrations with third-party health and smart kitchen devices, configure accessibility features, and set up sustainability-related alerts.

FIG. 8 is a block diagram illustrating the details of a digital processing system in which various aspects of the present disclosure are operative by execution of appropriate software instructions.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and so forth, herein do not denote any order, quantity, or importance, but rather are used to distinguish one element from another.

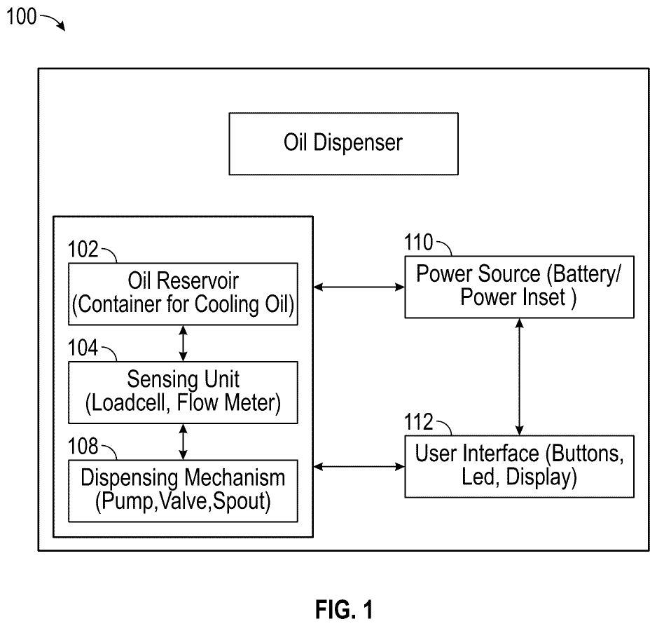

Referring to FIG. 1, which depicts a mechanical structural block diagram of the smart oil dispenser. It illustrates the primary physical components of the dispenser, including the oil reservoir, the integrated sensing unit for measurement, the dispensing mechanism, the protective housing, and the power source. An optional user interface is also shown for direct interaction.

The overall image, denoted by reference numeral 100, represents the complete smart oil dispenser system. This system is designed to provide precise control and monitoring over the dispensing of cooking oil, integrating various components to achieve its smart functionalities.

The Oil Reservoir 102 may comprise a container specifically designed for holding cooking oil. This reservoir may be refillable and constructed from materials suitable for food contact, such as glass, stainless steel, or food-grade plastic. The oil reservoir 102 may be functionally connected to the sensing unit 104 to allow for accurate measurement of the oil contained within or dispensed from it.

The Sensing Unit 104 may be integrated with the oil dispenser to measure the quantity of oil. This unit may comprise a load cell, which measures the weight of the oil reservoir 102 and its contents, thereby inferring the amount of oil dispensed by detecting changes in weight. Alternatively, or in addition, the sensing unit 104 may include a flow meter positioned within the dispensing path to directly measure the volume of oil flowing through. The sensing unit 104 may transmit its measurement data to an internal processing unit (not explicitly shown in this mechanical diagram but implied by the system's smart capabilities) for analysis and further action.

The Dispensing Mechanism 108 may be responsible for the controlled release of oil from the oil reservoir 102. This mechanism may include components such as a pump, a valve, or a precisely designed spout, which may be actuated to dispense oil in measured quantities. The dispensing mechanism 108 may receive control signals from the system's processing unit, which in turn may be influenced by data from the sensing unit 104 and user input. It may be functionally connected to the sensing unit 104 to ensure that the dispensed quantity is accurately measured.

The Power Source 110 may provide the necessary electrical energy for the operation of all active components within the smart oil dispenser. This may include an internal rechargeable battery for portability and convenience, or a power inlet for connection to an external power supply. The power source 110 may supply power to the sensing unit 104, the dispensing mechanism 108, and the user interface 112, ensuring continuous functionality of the device.

The User Interface 112 may provide a means for direct interaction between the user and the smart oil dispenser. This interface may include physical buttons for basic operations, LED indicators to display status or alerts, and/or a small display screen for showing dispensed quantities or other relevant information. The user interface 112 may allow users to initiate dispensing, adjust settings, or receive immediate feedback directly from the device. It may be functionally connected to the dispensing mechanism 108 for direct control and to the power source 110 for its operation.

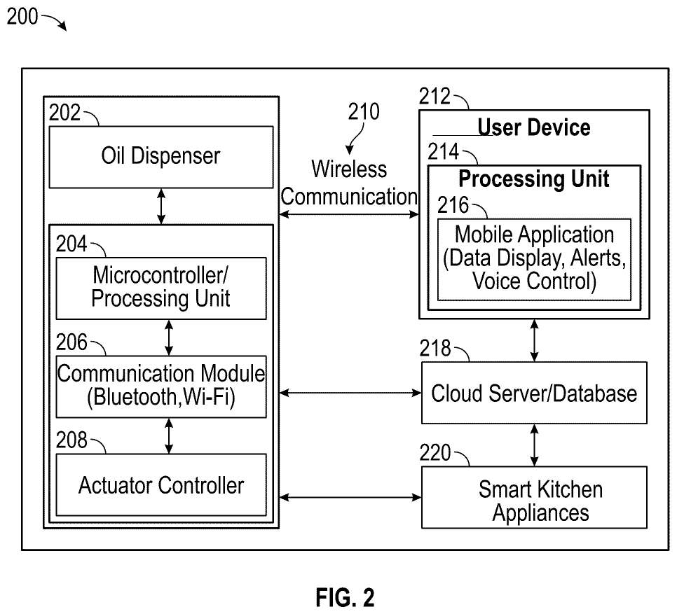

Referring to FIG. 2, which depicts a comprehensive system integration block diagram of the smart oil dispenser. It illustrates how the dispenser's hardware components (sensing unit, MCU, communication module, actuator) wirelessly connect to a user's mobile device running the application. This application, in turn, interacts with a cloud server for advanced functionalities and further integrates with third-party health applications and other smart kitchen appliances.

The overall image, denoted by reference numeral 200, represents the entire integrated ecosystem of the smart oil dispenser system. This encompasses the physical dispenser, the user's mobile device, cloud infrastructure, and various external smart home components, all working in concert to provide an intelligent oil management solution.

The Oil Dispenser 202 represents the physical hardware component of the smart oil dispenser, as further detailed in FIG. 1. This unit may include the oil reservoir, sensing unit (e.g., load cell, flow meter), and dispensing mechanism. It may be configured to measure dispensed oil quantities and receive commands for dispensing.

The Microcontroller/Processing Unit 204 may be the central computational brain of the oil dispenser 202. It may receive data from the sensing unit (not explicitly numbered here but part of the Oil Dispenser 202's internal components), process dispensing commands, and manage the overall operation of the dispenser. It may be functionally connected to the Communication Module 206 and the Actuator Controller 208, orchestrating data flow and control signals within the dispenser.

The Communication Module 206 may enable wireless data exchange between the oil dispenser 202 and external devices. This module may support various wireless communication protocols, such as Bluetooth for short-range direct communication or Wi-Fi for broader network connectivity. It may be bidirectionally connected to the Microcontroller/Processing Unit 204 to send usage data and receive commands.

The Actuator Controller 208 may be responsible for managing the physical dispensing mechanism of the oil dispenser 202. It may receive instructions from the Microcontroller/Processing Unit 204 to activate or deactivate the pump, valve, or other components of the dispensing mechanism, thereby controlling the flow of oil.

The Wireless Communication 210 represents the wireless link established between the Oil Dispenser 202 and the User Device 212. This connection may utilize protocols like Bluetooth or Wi-Fi, enabling the seamless transmission of oil usage data from the dispenser to the mobile application and the sending of control commands from the application back to the dispenser.

The User Device 212 may be a smartphone, tablet, or other portable electronic device utilized by the user. This device serves as the primary interface for interacting with the smart oil dispenser system, hosting the mobile application and displaying relevant information.

The Processing Unit 214 within the User Device 212 is responsible for executing the Mobile Application 216 and handling its computational tasks. This includes processing received data, rendering the user interface, and managing communication with the Cloud Server/Database 218 and potentially other Smart Kitchen Appliances 220.

The Mobile Application 216 may be a software application installed on the User Device 212. It may provide functionalities such as displaying real-time oil usage data, generating alerts for excessive consumption, allowing user input for dispensing quantities, and potentially incorporating voice control capabilities. It may process data received via Wireless Communication 210 and interact with the Cloud Server/Database 218.

The Cloud Server/Database 218 may represent a remote computing and storage infrastructure. It may store historical oil usage data, host learning algorithms for personalized suggestions, and manage integrations with third-party services. It may receive data from and send data to the Mobile Application 216, acting as a central hub for data management and advanced processing.

The Smart Kitchen Appliances 220 may include other intelligent devices found in a modern kitchen, such as smart scales, smart ovens, or other IoT-enabled cooking tools. These appliances may communicate with the Cloud Server/Database 218, enabling synergistic functionalities like automated oil dispensing based on recipe requirements or ingredient weights, thereby enhancing the overall smart kitchen ecosystem.

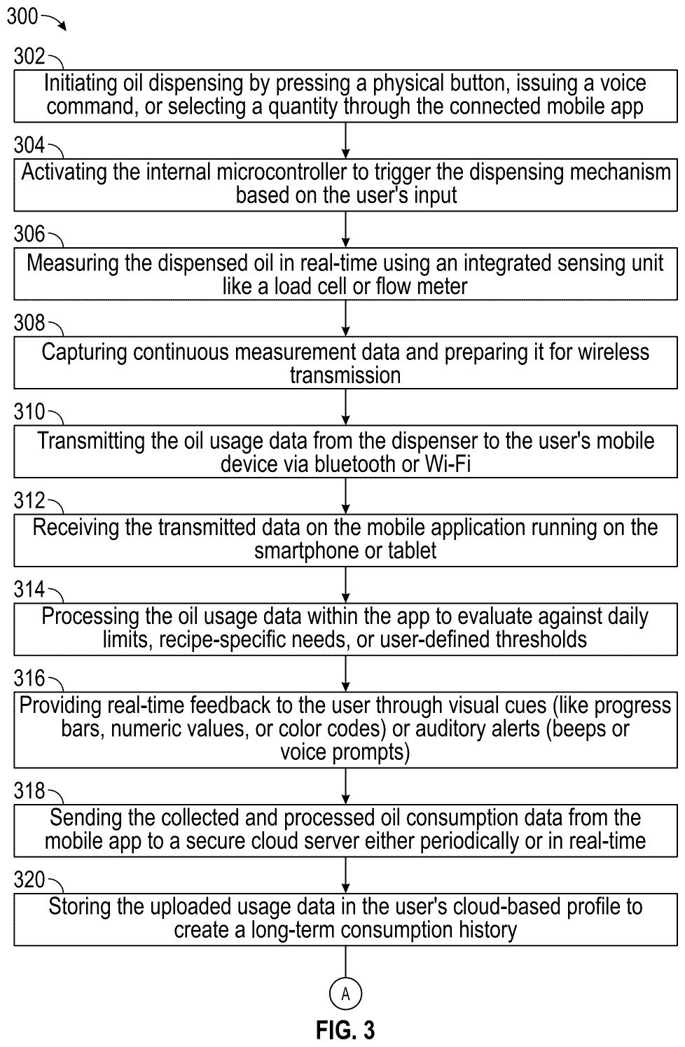

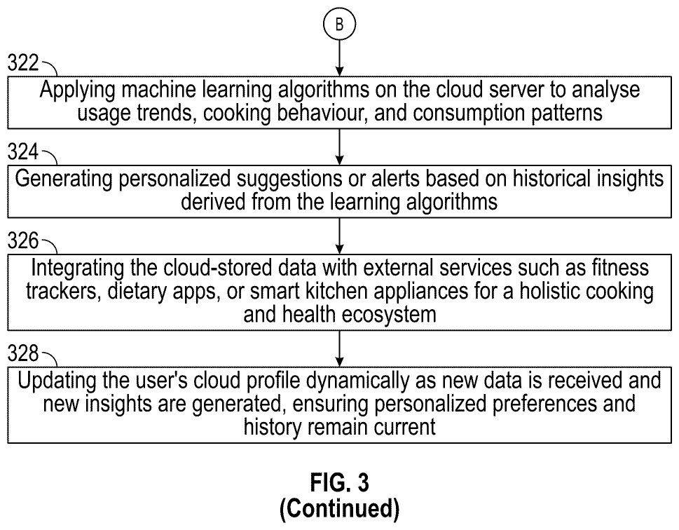

Referring to FIG. 3, which depicts an overall functional flow diagram of the smart oil dispenser system. It illustrates the sequence of operations from user interaction and oil dispensing to data transmission, processing by the mobile application, and subsequent cloud-based functionalities including data storage, learning algorithms, and integration with external services.

The overall image, denoted by reference numeral 300, represents the complete functional workflow of the smart oil dispenser system, detailing the interactions between the user, the physical dispenser, the mobile application, and the cloud infrastructure.

The step of 302: Initiating oil dispensing by pressing a physical button, issuing a voice command, or selecting a quantity through the connected mobile app, marks the beginning of the dispensing process. This step may involve a user physically interacting with the dispenser, speaking a command that is recognized by the dispenser or the mobile application, or inputting a desired quantity directly into the mobile application interface.

The step of 304: Activating the internal microcontroller to trigger the dispensing mechanism based on the user's input, follows the initiation. Upon receiving a valid dispense command, the dispenser's internal microcontroller may process this input and send appropriate signals to the actuator controller, which in turn may activate the dispensing mechanism (e.g., a pump or valve) to begin releasing oil.

The step of 306: Measuring the dispensed oil in real-time using an integrated sensing unit like a load cell or flow meter, occurs concurrently with the dispensing. As oil is released, the integrated sensing unit (e.g., a load cell detecting weight change or a flow meter measuring volume) may continuously monitor the exact quantity of oil being dispensed. This real-time measurement is crucial for accuracy and feedback.

The step of 308: Capturing continuous measurement data and preparing it for wireless transmission, involves the dispenser's processing unit collecting the raw data from the sensing unit 306. This data may then be formatted and prepared for efficient wireless transmission to the connected user device.

The step of 310: Transmitting the oil usage data from the dispenser to the user's mobile device via Bluetooth or Wi-Fi, involves the dispenser's communication module sending the prepared usage data wirelessly. This ensures that the mobile application on the user's device receives timely updates on oil consumption.

The step of 312: Receiving the transmitted data on the mobile application running on the smartphone or tablet, involves the mobile application actively listening for and acquiring the oil usage data sent from the dispenser. This data forms the foundation for the application's subsequent analytical and feedback functions.

The step of 314: Processing the oil usage data within the app to evaluate against daily limits, recipe-specific needs, or user-defined thresholds, involves the mobile application's internal logic analyzing the received data. It may compare the dispensed quantity against various criteria, such as a user's set daily oil intake limit, the recommended oil amount for a specific recipe being followed, or other personalized thresholds, to determine if the usage is appropriate.

The step of 316: Providing real-time feedback to the user through visual cues (like progress bars, numeric values, or color codes) or auditory alerts (beeps or voice prompts), is a critical user-facing function. Based on the processing in step 314, the mobile application may immediately inform the user about their oil consumption. This feedback may be displayed visually on the screen or provided audibly, particularly if an overuse warning is triggered.

The step of 318: Sending the collected and processed oil consumption data from the mobile app to a secure cloud server either periodically or in real-time, involves the mobile application transmitting the usage information to a remote server. This ensures data persistence, accessibility across multiple devices, and enables more complex, server-side computations.

The step of 320: Storing the uploaded usage data in the user's cloud-based profile to create a long-term consumption history, involves the cloud server receiving and securely saving the transmitted data. This creates a comprehensive historical record of the user's oil consumption, allowing for trend analysis and long-term tracking.

The step of 322: Applying machine learning algorithms on the cloud server to analyze usage trends, cooking behaviour, and consumption patterns, involves sophisticated computational processes on the server-side. These algorithms may analyze the accumulated historical data to identify recurring patterns, user preferences, and correlations between oil usage and other factors (e.g., time of day, meal type).

The step of 324: Generating personalized suggestions or alerts based on historical insights derived from the learning algorithms, is where the intelligence of the system becomes apparent. Based on the analysis from step 322, the cloud server may formulate tailored recommendations for oil usage, suggest healthier alternatives, or provide proactive alerts to the user, which are then pushed back to the mobile application.

The step of 326: Integrating the cloud-stored data with external services such as fitness trackers, dietary apps, or smart kitchen appliances for a holistic cooking and health ecosystem, involves the cloud server acting as a central data exchange point. It may facilitate the sharing of oil consumption data with other health-related applications or smart kitchen devices, enabling a more unified and comprehensive approach to health and cooking management.

The step of 328: Updating the user's cloud profile dynamically as new data is received and new insights are generated, ensuring personalized preferences and history remain current, ensures the system remains adaptive and relevant. As the user continues to use the dispenser and interact with the application, their cloud profile may be continuously refreshed with the latest usage data, updated personalized settings, and new insights derived from the learning algorithms, maintaining a highly customized experience.

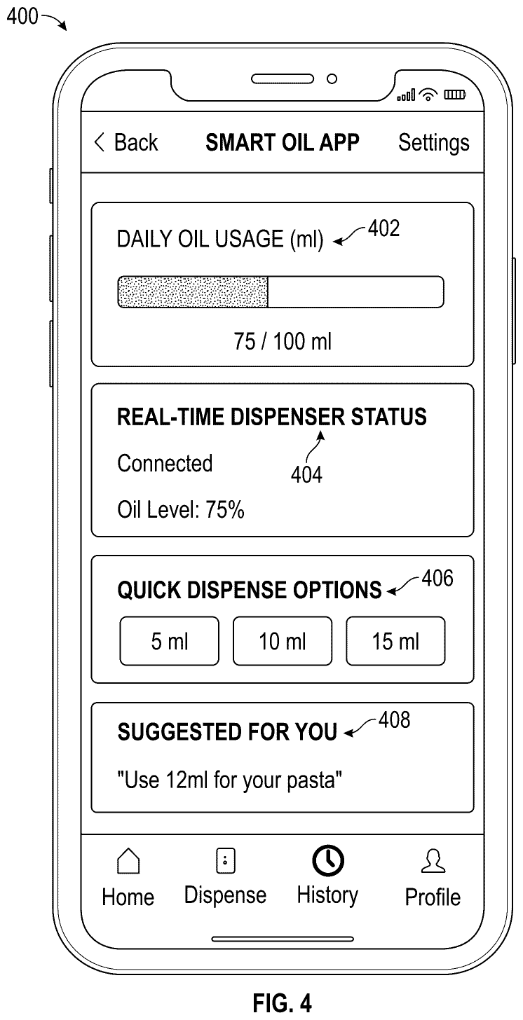

Referring to FIG. 4, which depicts a sample mobile application main dashboard screen. It provides an overview of daily oil usage, real-time dispenser status, quick dispense options, and personalized suggestions, serving as the central hub for user interaction.

The overall image, denoted by reference numeral 400, illustrates the graphical user interface of the mobile application as displayed on a user's smartphone or similar device. This screen serves as the primary entry point for users to monitor and interact with their smart oil dispenser system.

The Daily Oil Usage 402 section may prominently display the user's current oil consumption for the day, typically in milliliters (ml), against a predefined or personalized daily limit. This section may include a visual progress bar that intuitively indicates how close the user is to reaching their daily limit, providing immediate feedback on their consumption habits. This feature directly supports the objective of tracking oil usage and providing warnings.

The Real-time Dispenser Status 404 section may provide immediate information regarding the connectivity and operational state of the physical oil dispenser. It may indicate whether the dispenser is “Connected” to the mobile application and may display the current oil level remaining in the dispenser's reservoir (e.g., as a percentage). This ensures the user is aware of the dispenser's readiness and oil supply.

The Quick Dispense Options 406 section may offer convenient, pre-set buttons for common oil quantities (e.g., 5 ml, 10 ml, 15 ml). Tapping one of these buttons may send an immediate command to the dispenser to dispense the corresponding amount of oil, streamlining the dispensing process for frequently used quantities. This feature enhances the ease of use and direct control over the dispenser.

The Suggested for You 408 section may present personalized oil dispensing recommendations to the user. These suggestions may be generated by the learning algorithms on the cloud server, based on the user's historical cooking patterns, recipe preferences, and health goals. For example, it might suggest “Use 12 ml for your pasta,” providing intelligent guidance for healthier cooking. This directly aligns with the objective of applying learning algorithms to adjust oil suggestions based on user habits.

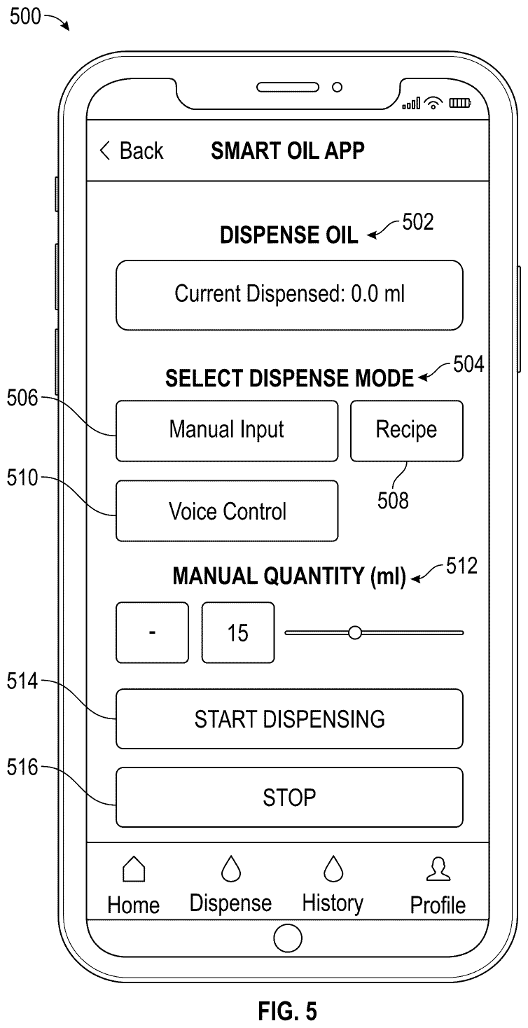

Referring to FIG. 5, which depicts a sample mobile application dispensing control screen. This screen allows users to select dispensing modes (manual, recipe, voice), input desired oil quantities, and initiate or stop the dispensing process, providing real-time feedback during operation.

The overall image, denoted by reference numeral 500, illustrates the graphical user interface of the mobile application specifically designed for controlling the oil dispensing process. This screen provides granular control and various methods for users to specify and manage oil output.

The Dispense Oil 502 section may prominently display the “Current Dispensed” quantity, showing the amount of oil that has been dispensed during the current operation, typically in milliliters (ml). This provides immediate visual feedback to the user as the oil is being dispensed. The Select Dispense Mode 504 section may offer different modes for controlling the oil dispensing. These modes allow users to choose their preferred method of inputting the desired oil quantity, enhancing flexibility and user experience.

The Manual Input 506 button, within the “Select Dispense Mode” section, may allow the user to manually enter a specific quantity of oil to be dispensed. This mode is suitable for users who know the exact amount of oil they require. The Recipe 508 button, also within the “Select Dispense Mode” section, may enable the user to select a recipe from a database. Upon selection, the application may automatically suggest or set the appropriate oil quantity based on the recipe's requirements, aligning with the objective of recipe integration.

The Voice Control 510 button, located within the “Select Dispense Mode” section, may activate the voice recognition feature. This allows users to verbally command the dispenser to dispense a specific quantity of oil, providing a hands-free operation option that supports accessibility objectives. The Manual Quantity (ml) 512 section may provide an interface for the user to specify the exact amount of oil to be dispensed when “Manual Input” mode is selected. This may include increment/decrement buttons and a slider for fine-tuning the desired quantity, ensuring precise control.

The Start Dispensing 514 button may initiate the oil dispensing process. Upon activation, the mobile application may send a command to the smart oil dispenser to begin releasing oil according to the selected mode and quantity. The Stop 516 button may immediately halt the oil dispensing process. This provides the user with an emergency override or the ability to stop dispensing at any point during the operation, ensuring control and preventing over-dispensing.

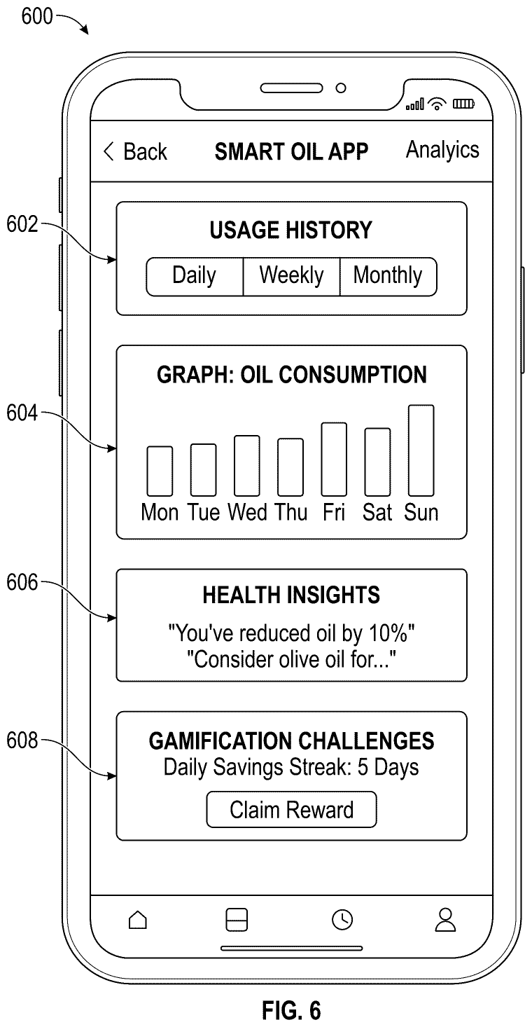

Referring to FIG. 6, which depicts a sample mobile application usage history and analytics screen. It presents historical oil consumption data through charts, offers personalized health insights based on usage patterns, and displays progress in gamification challenges to encourage healthier habits. The overall image, denoted by reference numeral 600, illustrates the graphical user interface of the mobile application dedicated to providing users with insights into their historical oil consumption and promoting healthier habits through data visualization and gamification.

The Usage History 602 section may allow users to view their oil consumption data over different timeframes, such as “Daily,” “Weekly,” or “Monthly.” This feature provides flexibility for users to analyze their habits at various granularities, supporting the objective of tracking oil usage. The Graph: Oil Consumption 604 section may visually represent the user's oil consumption over the selected period, typically as a bar chart showing daily or weekly usage. This graphical representation may make it easier for users to identify trends, peaks, and reductions in their oil intake, thereby aiding in understanding and managing their consumption patterns.

The Health Insights 606 section may provide personalized feedback and recommendations related to the user's oil consumption. These insights may be generated by machine learning algorithms analyzing the user's historical data and may include positive reinforcement (e.g., “You've reduced oil by 10%”) or suggestions for healthier choices (e.g., “Consider olive oil for . . . ”). This directly supports the objective of integrating with health apps and suggesting healthier oils. The Gamification Challenges 608 section may display progress in various challenges designed to encourage healthier oil consumption habits. This may include streaks for consistent healthy usage (e.g., “Daily Savings Streak: 5 Days”) and may offer options to “Claim Reward” for achieving milestones, thereby engaging users and promoting sustained behavioral change, aligning with the objective of gamification.

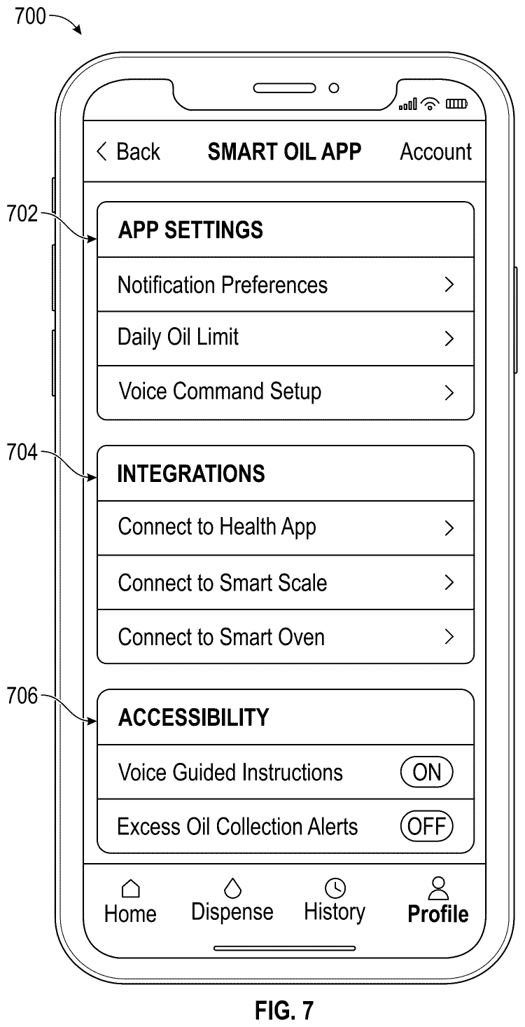

Referring to FIG. 7, which depicts a sample mobile application settings and integration screen. This screen allows users to customize app preferences, manage integrations with third-party health and smart kitchen devices, configure accessibility features, and set up sustainability-related alerts. The overall image, denoted by reference numeral 700, illustrates the graphical user interface of the mobile application's settings and configuration screen. This screen provides users with extensive control over the application's behavior, privacy, and connectivity with other smart devices and services.

The APP Settings 702 section may allow users to customize various operational parameters of the mobile application. This may include “Notification Preferences” to manage alerts and reminders, “Daily Oil Limit” to set personalized consumption targets, and “Voice Command Setup” to configure and train the voice control features for hands-free operation. This section directly supports the personalization and control objectives of the invention. The Integrations 704 section may provide options for connecting the smart oil dispenser system with external third-party applications and smart devices. This may include options to “Connect to Health App” for synchronizing oil consumption data with fitness trackers or dietary applications, “Connect to Smart Scale” for recipe-based dispensing, and “Connect to Smart Oven” for automated oil adjustments during cooking. This feature is crucial for achieving the objective of creating a holistic cooking and health ecosystem.

The Accessibility 706 section may offer features designed to enhance the usability of the smart oil dispenser for a wider range of users. This may include a toggle for “Voice Guided Instructions,” which provides auditory cues and confirmations for operations, and “Excess Oil Collection Alerts,” which notifies users about accumulated excess oil for reuse or proper disposal, supporting both accessibility and sustainability objectives.

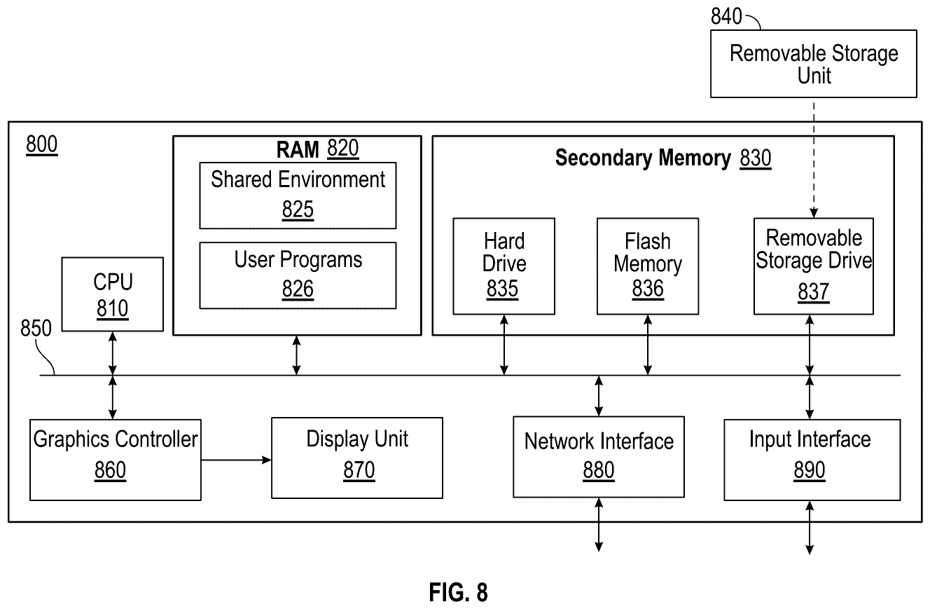

Referring to FIG. 8 is a block diagram 800 illustrating the details of a digital processing system 800 in which various aspects of the present disclosure are operative by execution of appropriate software instructions. The Digital processing system 800 may correspond to the computing device (or any other system in which the various features disclosed above can be implemented).

Digital processing system 800 may contain one or more processors such as a central processing unit (CPU) 810, random access memory (RAM) 820, secondary memory 830, graphics controller 860, display unit 870, network interface 880, and input interface 890. All the components except display unit 870 may communicate with each other over communication path 850, which may contain several buses as is well known in the relevant arts. The components of FIG. 8 are described below in further detail.

CPU 810 may execute instructions stored in RAM 820 to provide several features of the present disclosure. CPU 810 may contain multiple processing units, with each processing unit potentially being designed for a specific task. Alternatively, CPU 810 may contain only a single general-purpose processing unit.

RAM 820 may receive instructions from secondary memory 830 using communication path 850. RAM 820 is shown currently containing software instructions, such as those used in threads and stacks, constituting shared environment 825 and/or user programs 826. Shared environment 825 includes operating systems, device drivers, virtual machines, etc., which provide a (common) run time environment for execution of user programs 826.

Graphics controller 860 generates display signals (e.g., in RGB format) to display unit 870 based on data/instructions received from CPU 810. Display unit 870 contains a display screen to display the images defined by the display signals. Input interface 890 may correspond to a keyboard and a pointing device (e.g., touchpad, mouse) and may be used to provide inputs. Network interface 880 provides connectivity to a network (e.g., using Internet Protocol), and may be used to communicate with other systems (such as those shown in FIG. 1) connected to the network.

Secondary memory 830 may contain hard drive 835, flash memory 836, and removable storage drive 837. Secondary memory 830 may store the data software instructions (e.g., for performing the actions noted above with respect to the Figures), which enable digital processing system 800 to provide several features in accordance with the present disclosure.

Some or all of the data and instructions may be provided on removable storage unit 840, and the data and instructions may be read and provided by removable storage drive 837 to CPU 810. Floppy drive, magnetic tape drive, CD-ROM drive, DVD Drive, Flash memory, removable memory chip (PCMCIA Card, EEPROM) are examples of such removable storage drive 837.

Removable storage unit 840 may be implemented using medium and storage format compatible with removable storage drive 837 such that removable storage drive 837 can read the data and instructions. Thus, removable storage unit 840 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 840 or hard disk installed in hard drive 835. These computer program products are means for providing software to digital processing system 800. CPU 810 may retrieve the software instructions and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 830. Volatile media includes dynamic memory, such as RAM 820. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 850. Transmission media can also take the form of acoustic or light waves, such as those generated during radio-wave and infra-red data communications.

According to the non-limiting exemplary embodiments of the present invention, a system for dispensing and managing oil is provided, designed to offer precise control and intelligent monitoring of cooking oil consumption. This system may comprise a dispenser unit, which may include an oil reservoir configured to contain cooking oil. An integral sensing unit may be operably coupled to this oil reservoir, and may be configured to accurately measure the quantity of oil dispensed from the reservoir. Furthermore, a dispensing mechanism may be operably coupled to both the oil reservoir and the sensing unit, and may be configured to precisely release measured quantities of oil. The dispenser unit may also house a microcontroller, operably coupled to the sensing unit and the dispensing mechanism, which may be configured to process the measurement data received from the sensing unit and to control the operation of the dispensing mechanism. A communication module, operably coupled to the microcontroller, may be present to wirelessly transmit the collected oil usage data. The system may further include a user device, which may comprise a processing unit and a mobile application. This mobile application may be configured to receive the oil usage data transmitted from the dispenser's communication module, process this data by evaluating it against predefined thresholds, and subsequently provide real-time feedback to a user.

Additionally, the system may incorporate a cloud server, communicatively coupled to the user device, which may be configured to store historical oil usage data. This cloud server may be further configured to apply sophisticated machine learning algorithms to analyze the stored usage trends and generate personalized suggestions or alerts, whereby the entire system may enable precise monitoring and intelligent management of oil consumption, promoting healthier habits and reducing waste.

In an exemplary embodiment, the sensing unit of the dispenser may comprise at least one of a load cell, which may be configured to measure the weight of the oil reservoir and its contents, thereby inferring the dispensed amount through changes in weight, or a flow meter, which may be configured to directly measure the volume of oil flowing through the dispensing mechanism. This dual or singular sensing capability may ensure high accuracy in oil measurement.

In another exemplary embodiment, the communication module may be configured to establish wireless communication utilizing at least one of standard Bluetooth or Wi-Fi protocols. These protocols may enable robust and reliable data transmission between the dispenser unit and the user device, facilitating seamless connectivity.

In yet another exemplary embodiment, the mobile application may be further configured to provide real-time feedback to the user through various intuitive means. This feedback may include visual cues, such as dynamic progress bars illustrating daily consumption, numeric values indicating precise quantities, or color codes to signify usage status. Additionally, auditory alerts, such as distinct beeps or clear voice prompts, may be employed to notify the user, especially in instances of potential overuse.

According to a further exemplary embodiment, the cloud server may be configured to enhance user experience by generating personalized suggestions or alerts. This may be achieved by applying the machine learning algorithms to meticulously analyze the user's historical oil usage data and cooking behavior patterns. The mobile application may then be configured to prominently display these personalized suggestions or alerts to the user, offering tailored guidance for healthier oil consumption.

In an additional exemplary embodiment, the cloud server may be further configured to facilitate comprehensive integration with external health applications. This integration may allow for the seamless synchronization of the user's oil consumption data with their overall dietary intake records within various fitness trackers or dietary management applications, providing a holistic view of their nutritional habits.

In another exemplary embodiment, the cloud server may be further configured to integrate with other smart kitchen appliances. This may include connectivity with smart scales, enabling the system to suggest or automatically dispense precise oil amounts based on the weight of other ingredients in a recipe. It may also include integration with smart ovens, allowing for automated oil adjustments in accordance with specific cooking programs, thereby creating a more interconnected and efficient kitchen environment.

Furthermore, in an exemplary embodiment, the mobile application may be configured to offer advanced user interaction features. This may include enabling voice control, allowing users to initiate dispensing and specify oil quantities hands-free. The mobile application may also be configured to provide voice-guided instructions, enhancing accessibility for users who may benefit from auditory cues during operation.

According to a non-limiting exemplary embodiment of the present invention, a method for dispensing and managing oil is provided, which may comprise the step of initiating oil dispensing via a user input to a smart oil dispenser. This may be followed by the step of activating, by a microcontroller within the smart oil dispenser, a dispensing mechanism based on the user's input. Subsequently, the method may involve the step of measuring, by a sensing unit integrated with the smart oil dispenser, a quantity of oil dispensed in real-time. The measured oil usage data may then be transmitted, by a communication module of the smart oil dispenser, wirelessly to a mobile application on a user device. Upon reception, the method may include the step of receiving, by the mobile application, the transmitted oil usage data. This data may then undergo the step of processing, by a processing unit of the user device, within the mobile application, where it is evaluated against at least one of daily limits, recipe-specific needs, or user-defined thresholds. Following processing, the method may involve the step of providing, by the mobile application, real-time feedback to the user based on the processed data. The collected and processed oil consumption data may then undergo the step of sending, by the mobile application, to a secure cloud server.

The cloud server may then perform the step of storing the uploaded usage data in a user's cloud-based profile to create a long-term consumption history. To enhance intelligence, the method may include the step of applying, by the cloud server, machine learning algorithms to analyze usage trends, cooking behavior, and consumption patterns from the stored data. Based on this analysis, the method may proceed with the step of generating, by the cloud server, personalized suggestions or alerts derived from the learning algorithms. Furthermore, the method may involve the step of integrating, by the cloud server, the cloud-stored data with external services including at least one of fitness trackers, dietary applications, or smart kitchen appliances, thereby fostering a holistic cooking and health ecosystem. Finally, the method may conclude with the step of updating, by the cloud server, the user's cloud profile dynamically as new data is received and new insights are generated, ensuring personalized preferences and history remain current and relevant.

In an exemplary embodiment of the method, the step of providing real-time feedback may include displaying visual cues such as progress bars or numeric values on the mobile application interface, or emitting auditory alerts such as beeps or voice prompts from the user device or dispenser. Additionally, the step of generating personalized suggestions may include recommending specific oil quantities for particular recipes or suggesting healthier oil alternatives based on user health goals and historical consumption patterns.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A system for dispensing and management of oil, comprising:
a dispenser unit including an oil reservoir configured to contain cooking oil;
a sensing unit, operably coupled to the oil reservoir, configured to measure a quantity of oil dispensed from the oil reservoir;
a dispensing mechanism, operably coupled to the oil reservoir and the sensing unit, configured to release measured quantities of oil;
a microcontroller, operably coupled to the sensing unit and the dispensing mechanism, configured to process measurement data and control the dispensing mechanism;
a communication module, operably coupled to the microcontroller, configured to wirelessly transmit oil usage data;
a user device including a processing unit and a mobile application, the mobile application configured to receive the oil usage data from the communication module, process the oil usage data against predefined thresholds, and provide real-time feedback to a user; and
a cloud server, communicatively coupled to the user device, configured to store historical oil usage data, wherein the cloud server is further configured to apply machine learning algorithms to analyze usage trends and generate personalized suggestions or alerts, whereby the system enables precise monitoring and management of oil consumption.

2. The system of claim 1, wherein the sensing unit comprises at least one of a load cell configured to measure a weight of the oil reservoir and its contents, or a flow meter configured to measure a volume of oil flowing through the dispensing mechanism.

3. The system of claim 1, wherein the communication module is configured to establish wireless communication via at least one of Bluetooth or Wi-Fi protocols.

4. The system of claim 1, wherein the mobile application is further configured to provide real-time feedback through at least one of visual cues, including progress bars, numeric values, or color codes, or auditory alerts, including beeps or voice prompts.

5. The system of claim 1, wherein the cloud server is further configured to generate personalized suggestions or alerts by analyzing historical oil usage data and cooking behavior using the machine learning algorithms, and wherein the mobile application is configured to display said personalized suggestions or alerts to the user.

6. The system of claim 1, wherein the cloud server is further configured to integrate the stored oil usage data with external health applications to synchronize oil consumption data with a user's overall dietary intake records.

7. The system of claim 1, wherein the cloud server is further configured to integrate with smart kitchen appliances, including at least one of smart scales or smart ovens, to enable automated oil dispensing based on recipe requirements or ingredient weights.

8. The system of claim 1, wherein the mobile application is further configured to enable voice control for initiating dispensing and specifying oil quantities, and wherein the mobile application is further configured to provide voice-guided instructions for enhanced accessibility.

9. A method for dispensing and managing oil, comprising:
initiating oil dispensing via a user input to a smart oil dispenser;
activating, by a microcontroller within the smart oil dispenser, a dispensing mechanism based on the user input;
measuring, by a sensing unit integrated with the smart oil dispenser, a quantity of oil dispensed in real-time;
transmitting, by a communication module of the smart oil dispenser, the measured oil usage data wirelessly to a mobile application on a user device;
receiving, by the mobile application, the transmitted oil usage data;
processing, by a processing unit of the user device, the oil usage data within the mobile application against at least one of daily limits, recipe-specific needs, or user-defined thresholds;
providing, by the mobile application, real-time feedback to the user based on the processed data;
sending, by the mobile application, the collected and processed oil consumption data to a cloud server;
storing, by the cloud server, the uploaded usage data in a user's cloud-based profile to create a long-term consumption history;
applying, by the cloud server, machine learning algorithms to analyze usage trends, cooking behavior, and consumption patterns from the stored data;
generating, by the cloud server, personalized suggestions or alerts based on historical insights derived from the learning algorithms;
integrating, by the cloud server, the cloud-stored data with external services including at least one of fitness trackers, dietary applications, or smart kitchen appliances; and
updating, by the cloud server, the user's cloud profile dynamically as new data is received and new insights are generated, thereby providing an intelligent and personalized oil management experience.

10. The method of claim 9, wherein providing real-time feedback includes displaying visual cues such as progress bars or numeric values, or emitting auditory alerts such as beeps or voice prompts, and wherein generating personalized suggestions includes recommending specific oil quantities for recipes or suggesting healthier oil alternatives.
